(19) United States
(12) Patent Application Publication
(10) Pub. No.: US 2021/0134411 A1
(43) Pub. Date: May 6, 2021

(54) INFORMATION PROCESSING DEVICE, INFORMATION PROCESSING METHOD, AND NON-TRANSITORY RECORDING MEDIUM ON WHICH PROGRAM FOR INFORMATION PROCESSING IS RECORDED

(71) Applicant: OMRON HEALTHCARE Co., Ltd., Kyoto (JP)

(72) Inventors: Toru DENO, Kyoto (JP); Hironori SATO, Kyoto (JP); Takahiro HAMAGUCHI, Kyoto (JP); Takanobu YAMAUCHI, Kyoto (JP); Yasushi MATSUOKA, Kyoto (JP)

(21) Appl. No.: 17/145,807

(22) Filed: Jan. 11, 2021

Related U.S. Application Data
(63) Continuation of application No. PCT/JP2019/026070, filed on Jul. 1, 2019.

(30) Foreign Application Priority Data
Jul. 18, 2018 (JP) 2018-134894

(51) Int. Cl. G16H 10/60; G16H 40/67

(52) U.S. Cl. CPC G16H 10/60; H04L 67/02; G16H 40/67

(57) ABSTRACT

Health-related information is provided suitable for a subject. An information processing device includes an acquisition unit that acquires first information indicating a subject's interest in health, an estimation unit that estimates a subject's degree of interest in health based on the first information, a selection unit that selects second information regarding health to be provided to the subject based on the degree of interest, and an output unit that outputs the second information.

### CROSS-REFERENCE TO RELATED APPLICATIONS

This application is the U.S. national stage application filed pursuant to 35 U.S.C. 365(c) and 120 as a continuation of International Patent Application No. PCT/JP2019/026070, filed Jul. 1, 2019, which application claims priority from Japanese Patent Application No. 2018-134894, filed Jul. 18, 2018, which applications are incorporated herein by reference in their entireties.

### TECHNICAL FIELD

The present invention relates to an information processing device, an information processing method, and a non-transitory recording medium on which a program for information processing is recorded.

### BACKGROUND ART

In recent years, various health-related technologies have been developed due to the growing consciousness for health among people.

Patent Document 1 discloses a technology for analyzing a user's health state and notifying the user of the analysis result of the current user's health state and health guidance information.

### CITATION LIST

### Patent Literature

Patent Document 1: JP 2002-63279 A

### SUMMARY OF INVENTION

### Technical Problem

However, the health guidance information disclosed in Patent Document 1 has the same content for a plurality of users in the same health state. Therefore, the content of the health guidance information is understandable for anyone, but may be unsatisfactory for some people.

The present invention has been made in view of the above circumstances, and an object of the present invention is to provide a technology for providing health-related information suitable for a subject.

### Solution to Problem

An information processing device according to a first aspect of the present disclosure includes an acquisition unit that acquires first information indicating a subject's interest in health, including information indicating the number of health-related apparatuses to be connected to a terminal associated with the subject, an estimation unit that estimates a subject's degree of interest in health based on the number of health-related apparatuses, a selection unit that selects second information regarding health to be provided to the subject based on the degree of interest, and an output unit that outputs the second information.

The desired content of health information differs between those who have a high degree of interest in health and those who have a low degree of interest in health. The information processing device can provide the health information appropriate for the subject based on the degree of interest of the subject. The subject can effectively utilize the health information appropriate for the subject.

People with the high degree of interest in health tend to use terminals for variety of purposes for their health. On the other hand, people with the low degree of interest in health tend to rarely use terminals for their health. The information processing device can improve the estimation accuracy of the degree of interest in health by referring to the actual state of health-related use by the terminal.

According to a second aspect of the present disclosure, in the information processing device according to the first aspect, the first information includes information indicating the number of accesses or a frequency of accesses to a health-related web page from the terminal, and the estimation unit estimates the degree of interest based on the number of accesses or the frequency of accesses.

People with the high degree of interest in health tend to access health-related web pages many times using the terminals. On the other hand, people with the low degree of interest in health tend to rarely access health-related web pages using the terminals. The information processing device can improve the estimation accuracy of the degree of interest in health by referring to the number of accesses or the frequency of accesses to the health-related web pages from the terminal.

According to a third aspect of the present disclosure, in the information processing device according to the first aspect, the first information includes information indicating the number of web pages or applications regarding health registered in the terminal, and the estimation unit estimates the degree of interest based on the number of web pages or applications.

People with the high degree of interest in health tend to register many URLs of health-related web pages in the browser used on the terminals. Also, people with the high degree of interest in health tend to install many health-related applications on the terminals. On the other hand, people with the low degree of interest in health tend to rarely register the URLs of health-related web pages in the browser used on the terminals. Also, people with the low degree of interest in health tend to rarely install health-related applications on the terminals. The information processing device can improve the estimation accuracy of the degree of interest in health by referring to the number of health-related web pages or applications registered in the terminal.

People with the high degree of interest in health tend to connect many health-related apparatuses to the terminals. Also, people with the high degree of interest in health tend to connect not only health-related apparatuses, such as a scale, that are widely used in homes, but also medical health-related apparatuses, such as a blood pressure monitor, to the terminals. It can be said that the medical health-related apparatuses, such as the blood pressure monitor, are apparatuses for people with high degree of interest in health than the health-related apparatuses, such as the scale, that are widely used in homes. On the other hand, people with the low degree of interest in health tend to rarely connect health-related apparatuses to the terminals. Also, people with the low degree of interest in health tend to connect health-related apparatuses, such as the scale, that are widely used in homes to the terminals, but not connect medical health-related apparatuses, such as the blood pressure monitor, to the terminals. The information processing device can improve the estimation accuracy of the degree of interest in health by referring to the number or type of health-related apparatuses connected to the terminal.

According to a fourth aspect of the present disclosure, in the information processing device according to the second or third aspect, the estimation unit creates a history indicating a shift of the degree of interest according to the estimation of the degree of interest based on the number of health-related apparatuses, and the selection unit detects a highest level of the degree of interest indicated in the history according to the estimation of the degree of interest based on the number of health-related apparatuses, and selects the second information at a level corresponding to the highest level of the degree of interest.

Even when the subject's interest in health is lower than before, the subject's level of knowledge regarding health remains high. The information processing device, by referring to the history indicating the shift of the degree of interest, can provide the health information with a degree of detail appropriate for the subject without degrading the quality of the health information as compared with the health information provided to the subject in the past. Since the subject receives the health information at the level that the subject can understand, the subject can effectively utilize the health information.

According to a fifth aspect of the present disclosure, in the information processing device according to the second or third aspect, the estimation unit creates a history indicating a shift of the degree of interest according to the estimation of the degree of interest based on the number of health-related apparatuses, and the selection unit determines whether the subject associated with the terminal is the same as before according to the estimation of the degree of interest based on the number of health-related apparatuses, and when a determination result indicating that the subject is the same as before, detects a highest level of the degree of interest indicated in the history and selects the second information at a level corresponding to the highest level of the degree of interest, and when a determination result indicating that the subject is not the same as before, selects the second information at or below the level corresponding to the degree of interest estimated based on the number of health-related apparatuses. The subject (owner) associated with the terminal may change due to the transfer of the terminal or the like. The knowledge level of the current subject associated with the terminal may be lower than the knowledge level of another subject previously associated with the terminal. Thus, the current subject associated with the terminal may not be able to understand the same level of health information that was previously provided to the other subject. The current subject associated with the terminal preferably receives health information at or below the level corresponding to the current subject's degree of interest. The information processing device can provide the health information with a degree of detail appropriate for the subject by changing the level of health information that is provided to the subject according to whether the subject associated with the terminal is the same as before.

According to a sixth aspect of the present disclosure, in the information processing device according to the first aspect, the acquisition unit acquires the first information from a first external apparatus, and the output unit outputs the second information to a second external apparatus different from the first external apparatus. For example, the subject may want to input the health-related content into the first external apparatus such as an artificial intelligence (AI) speaker or a smart speaker by voice, and confirm the health information on the second external apparatus such as the subject's portable terminal. With the separate apparatuses, the subject can input the health-related content and confirm the health information, thereby improving the convenience of the subject.

According to a seventh aspect of the present disclosure, in the information processing device according to any one of the first to sixth aspects, the selection unit selects the second information with an appropriate degree of detail according to the degree of interest.

The higher the subject's interest in health, the more likely the subject wants more detailed health information. The information processing device can provide the health information with the degree of detail appropriate for the subject based on the subject's degree of interest. The subject can effectively utilize the health information appropriate for the subject.

An information processing method according to an eighth aspect of the present disclosure includes an acquisition process for acquiring first information indicating a subject's interest in health, including information indicating the number of health-related apparatuses to be connected to a terminal associated with the subject, an estimation process for estimating a subject's degree of interest in health based on the number of health-related apparatuses, a selection process for selecting second information regarding health to be provided to the subject based on the degree of interest, and an output process for outputting the second information.

According to the eighth aspect, the information processing method can obtain the same effects as the first aspect described above.

In a non-transitory recording medium on which a program for information processing is recorded, according to a ninth aspect of the present disclosure, the program causes a computer to execute processing of each unit included in the information processing device according to any one of the first to seventh aspects. According to the ninth aspect, the non-transitory recording medium on which the program for information processing is recorded can obtain the same effects as the first aspect described above.

### Advantageous Effects of Invention

According to the present invention, a technology for providing health-related information suitable for a subject can be provided.

### BRIEF DESCRIPTION OF DRAWINGS

FIG. 1 is a diagram schematically illustrating an application example of a sever according to the present embodiment.

FIG. 2 is a diagram illustrating an overall configuration of an information distribution system according to the present embodiment.

FIG. 3 is a block diagram illustrating a hardware configuration of the server according to the present embodiment.

FIG. 4 is a block diagram illustrating a software configuration of the server according to the present embodiment.

FIG. 5 is a block diagram illustrating a hardware configuration of a portable terminal according to the present embodiment.

FIG. 6 is a flowchart showing an information output operation in the server according to the present embodiment.

### DESCRIPTION OF EMBODIMENTS

Hereinafter, an embodiment according to the present disclosure (hereinafter, also referred to as “the present embodiment”) will be described below with reference to the drawings. However, the present embodiment described below is merely illustrative in all respects. Note that elements that are the same as or similar to the elements described hereinafter are given the same or similar reference numerals, and duplicate descriptions will be omitted.

### 1 Application Example

The present embodiment is a technology for distributing health information regarding health suitable for each person based on interest information that indicates each person's interest in health.

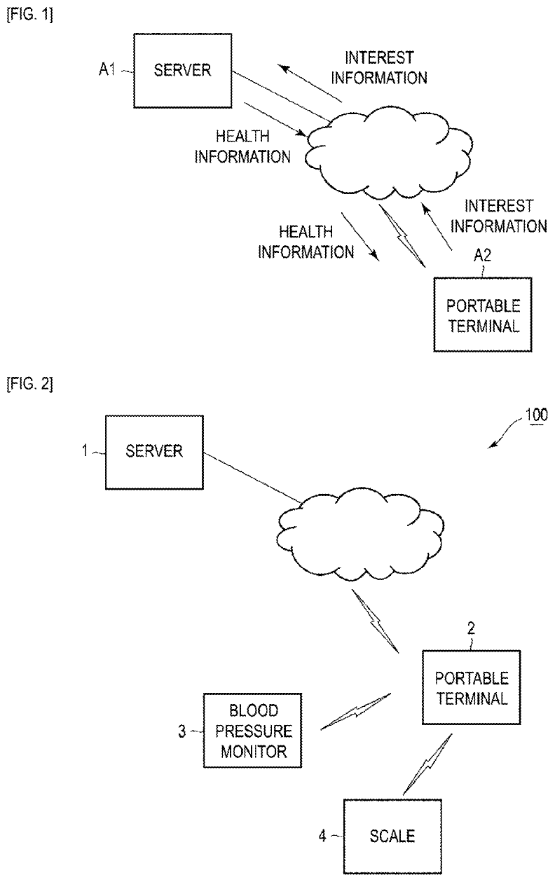

FIG. 1 is a diagram schematically illustrating an application example of a server A1.

The server A1 receives the interest information of the subject associated with a portable terminal A2 from the portable terminal A2 via a network. The server A1 estimates the subject's degree of interest in health based on the interest information. The server A1 selects the health information to be provided to the subject based on the degree of interest. The server A1 transmits the health information to the portable terminal A2. The subject can confirm the health information that is appropriate for the subject on the portable terminal A2.

In this way, the server A1 can provide the health information suitable for each person.

### 2 Configuration Example

### Information Distribution System

FIG. 2 is a diagram illustrating an overall configuration of an information distribution system 100. The information distribution system 100 is a system for distributing information regarding health suitable for each person based on information indicating each person's interest in health. The information indicating an interest in health is also referred to as interest information. The interest information is also referred to as first information. An example of the interest information will be described later. The information regarding health is also referred to as health information. The health information is also referred to as second information. The health information will be described later.

The information distribution system 100 includes a server 1 and a portable terminal 2. The server 1 and the portable terminal 2 can communicate with each other via a network. For example, the network is the Internet.

The server 1 is a device for distributing the health information suitable for each person based on the degree of interest of each person. The server 1 can estimate each person's degree of interest in health based on each person's interest information.

The portable terminal 2 is a device for transmitting the interest information of the subject associated with the portable terminal 2 to the server 1. The subject associated with the portable terminal 2 can also be considered the owner of the portable terminal 2. For example, the portable terminal 2 is, but is not limited to, a smart phone or a tablet. The portable terminal 2 communicates directly with a blood pressure monitor 3 and receives blood pressure data. The blood pressure monitor 3 is an apparatus to be connected to the portable terminal 2. Also, the portable terminal 2 communicates directly with a scale 4 and receives weight data. The scale 4 is an apparatus to be connected to the portable terminal 2. The blood pressure monitor 3 and the scale 4 are examples of health related apparatuses. The health-related apparatuses are related to the interest information of the subject, as described later.

### Server

### Hardware Configuration

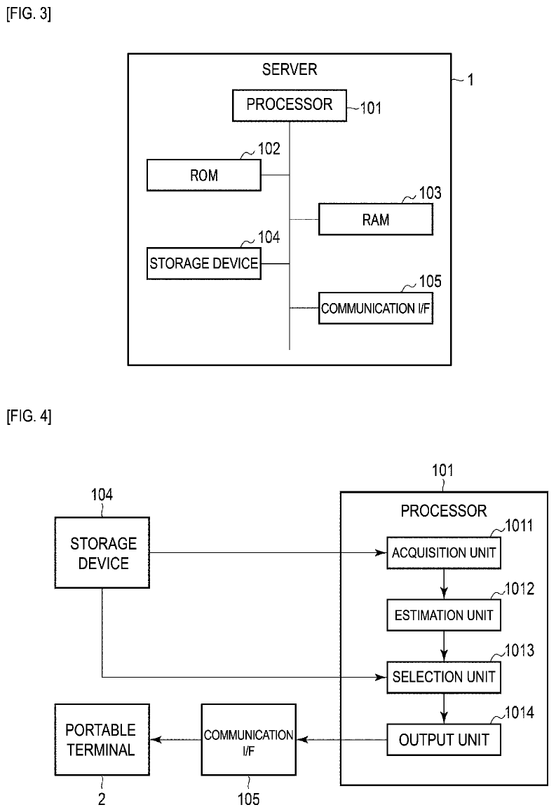

FIG. 3 is a block diagram illustrating a hardware configuration of the server 1. The server 1 includes a processor 101, a read only memory (ROM) 102, a random access memory (RAM) 103, a storage device 104, and a communication interface 105. The elements are electrically connected to each other. Note that, in FIG. 3, the communication interface is described as “communication I/F”.

The processor 101 controls each component of the server 1. For example, the processor 101 is a central processing unit (CPU), but is not limited thereto. The processor 101 deploys a program stored in the storage device 104 into the RAM 103 in order to execute the server 1. Then, the processor 101 interprets and executes the program deployed in the RAM 103, so that the processor 101 can execute various operations.

The storage device 104 is a so-called auxiliary storage device. For example, the storage device 104 is a hard disk drive (HDD), but is not limited thereto. The storage device 104 stores a program that is executed by the processor 101. Note that the program may be stored in the storage device 104 in advance. The program may be downloaded to the server 1 via the network. The program may be stored and distributed on a non-transitory computer-readable medium.

The storage device 104 stores the interest information received by the server 1 from each person's portable terminal 2. The server 1 may store the interest information in the storage device 104 based on the reception of the interest information autonomously transmitted by each person's portable terminal 2. Alternatively, the server 1 may transmit a request for the interest information to each person's portable terminal 2, and store the interest information in the storage device 104 based on the reception of the interest information from the portable terminal 2.

An example of the interest information will be described. For example, the interest information includes information indicating the health-related use by the portable terminal 2 associated with each person. Information indicating the health-related use by the portable terminal 2 is also referred to as usage information. The usage information is information indicating a value based on the health-related use in the portable terminal 2. Note that the interest information may be any information indicating each person's interest in health, and is not limited to the usage information.

The usage information is exemplified below, but the usage information is not limited to the following examples.

In one example, the usage information includes information indicating the number of accesses or the frequency of accesses to the health-related web pages from the portable terminal 2. The health-related web page can be any web page that has the potential to lead to health. The range included in the health-related web page can be arbitrarily set. The health-related web page may include a web page about medical. The health-related web page may include a web page about medicine. The health-related web page may include a web page about disease. The health-related web page may include a web page about exercise. The health-related web page may include a web page about the health-related apparatus such as the blood pressure monitor or the scale. The number of accesses may be the number of accesses within a predetermined time period. The number of accesses may be the number of accesses from the start of operation of the portable terminal 2 to the present. Multiple accesses to the same web page may be counted as one access. The number of accesses may be counted as the total number of accesses even when the same web page is accessed multiple times. The frequency of accesses may be a frequency of accesses within a predetermined time period or a frequency of accesses from the start of operation of the portable terminal 2 to the present. The access frequency may be an average time interval for accessing health-related web pages. The access frequency may be the ratio of the health-related web pages to all the web pages accessed by each person.

In another example, the usage information includes information indicating the number of health-related web pages or applications registered in the portable terminal 2. For example, the number of health-related web pages registered in the portable terminal 2 corresponds to the number of uniform resource locators (URLs) of health-related web pages registered in the browser used in the portable terminal 2. For example, the number of health-related applications registered in the portable terminal 2 corresponds to the number of health-related applications installed in the portable terminal 2.

In yet another example, the usage information includes information indicating the number or type of health-related apparatuses to be connected to the portable terminal 2. For example, the number of health-related apparatuses to be connected to the portable terminal 2 corresponds to the number of applications installed in the portable terminal 2 to enable communication between the portable terminal 2 and the health-related apparatuses. When an application for enabling communication with the blood pressure monitor 3 and an application for enabling communication with the scale 4 are installed in the portable terminal 2, the number of health-related apparatuses to be connected to the portable terminal 2 is two. For example, the types of health-related apparatuses connected to the portable terminal 2 correspond to the types of applications installed in the portable terminal 2 to enable communication between the portable terminal 2 and the health-related apparatuses. When the application for enabling communication with the blood pressure monitor 3 is installed in the portable terminal 2, the type of health-related apparatus to be connected to the portable terminal 2 includes the blood pressure monitor. When the application for enabling communication with the scale 4 is installed in the portable terminal 2, the type of health-related apparatus to be connected to the portable terminal 2 includes a scale.

The storage device 104 stores the health information. For example, the storage device 104 stores the pieces of health information with an appropriate degree of detail. The degree of detail also corresponds to the degree of difficulty or the degree of specialty. For example, there are three degrees of detail. Health information with a high degree of detail is information at a level that can be understood by a person who has a lot of knowledge. For example, the health information with the high degree of detail may be detailed information about a medicine that is effective against high blood pressure, such as the components of the medicine and the mechanism of action of the medicine. Health information with a medium degree of detail is information at a level that can be understood by a certain number of people. For example, the health information with the medium degree of detail may be information such as a method for health in daily life for preventing high blood pressure. The health information with a low degree of detail is information at a level that can be understood by the majority of people. For example, the health information with the low degree of detail may be schematic information describing what high blood pressure is. Note that the degree of detail may be divided into two degrees or four degrees or more.

The communication interface 105 is an interface that communicably connects the server 1 to another device. The communication interface 105 may include an interface for wired communication, or may include an interface for wireless communication, such as mobile communication (3G, 4G, or the like) and WLAN.

Note that, with regard to a specific hardware configuration of the server 1, components can be omitted, replaced, and added as appropriate according to the embodiment. For example, the server 1 may include a plurality of processors.

### Software Configuration

FIG. 4 is a block diagram illustrating a software configuration of the server 1. The processor 101 implements an acquisition unit 1011, an estimation unit 1012, a selection unit 1013, and an output unit 1014.

The acquisition unit 1011 will be described. The acquisition unit 1011 acquires the interest information of the subject as exemplified below. The acquisition unit 1011 acquires the subject's interest information stored in the storage device 104. The acquisition unit 1011 outputs the interest information of the subject to the estimation unit 1012. Each person's interest information stored in the storage device 104 is sequentially outputted to the estimation unit 1012. The acquisition unit 1011 may execute a process for acquiring the interest information at any timing. The acquisition unit 1011 may execute the process for acquiring the interest information at regular intervals.

The estimation unit 1012 will be described. The estimation unit 1012 estimates the subject's degree of interest in health based on the interest information of the subject, as exemplified below. First, the estimation unit 1012 receives the interest information of the subject from the acquisition unit 1011. Next, the estimation unit 1012 refers to the interest information of the subject. For example, the estimating unit 1012 can estimate the degree of interest by using a value based on one or more pieces of information included in the interest information. The estimation unit 1012 may estimate the degree of interest by inputting the value based on the one or more pieces of information included in the interest information into a predetermined calculation equation. The estimation unit 1012 may give a weighting factor to the value based on the one or more pieces of information included in the interest information according to a predetermined rule.

The estimation unit 1012 may refer to the usage information included in the interest information of the subject. The estimation unit 1012 can estimate the subject's degree of interest in health based on the health-related use by the portable terminal 2 indicated by the usage information. The more the subject uses the portable terminal 2 regarding health, the higher the degree of interest is estimated by the estimation unit 1012. For example, the estimation unit 1012 can estimate the degree of interest by using a value based on one or more pieces of information included in the usage information. The estimation unit 1012 may estimate the degree of interest by inputting the value based on the one or more pieces of information included in the usage information into a predetermined calculation equation. The estimation unit 1012 may give a weighting factor to the value based on the one or more pieces of information included in the usage information according to a predetermined rule.

The estimation unit 1012 may refer to the information indicating the number of accesses or the frequency of accesses to the health-related web pages from the portable terminal 2 included in the usage information. The estimation unit 1012 can estimate the subject's degree of interest in health based on the number of accesses or the frequency of accesses to the health-related web pages from the portable terminal 2. The more the number of accesses or frequency of accesses to the health-related web pages from the portable terminal 2, the higher the degree of interest is estimated by the estimation unit 1012. In one example, the estimation unit 1012 may estimate the degree of interest by putting the number of accesses or the frequency of accesses into a predetermined calculation equation. In another example, the estimation unit 1012 may refer to a table in which a predetermined number of accesses and the degree of interest are associated with each other, and estimate the degree of interest based on the number of accesses. Similarly, the estimation unit 1012 may refer to a table in which a predetermined frequency of accesses and the degree of interest are associated with each other, and estimate the degree of interest based on the frequency of accesses. These tables are stored in the storage device 104.

The estimation unit 1012 may refer to the information indicating the number of health-related web pages or applications registered in the portable terminal 2 included in the usage information. The estimation unit 1012 can estimate the subject's degree of interest in health based on the number of health-related web pages or applications registered in the portable terminal 2. The more the number of health-related web pages or applications registered in the portable terminal 2, the higher the degree of interest is estimated by the estimation unit 1012. In one example, the estimation unit 1012 may estimate the degree of interest by putting the number of web pages or applications into a predetermined calculation equation. In another example, the estimation unit 1012 may refer to a table in which a predetermined number of web pages and the degree of interest are associated with each other, and estimate the degree of interest based on the number of web pages. Similarly, the estimation unit 1012 may refer to a table in which a predetermined number of applications and the degree of interest are associated with each other, and estimate the degree of interest based on the number of applications. These tables are stored in the storage device 104.

The estimation unit 1012 may refer to information indicating the number or type of health-related apparatuses to be connected to the portable terminal 2 included in the usage information. The estimation unit 1012 can estimate the subject's degree of interest in health based on the number or type of health-related apparatuses to be connected to the portable terminal 2. The more the number of health-related apparatuses connected to the portable terminal 2, the higher the degree of interest is estimated by the estimation unit 1012. When a specific type (for example, the medical health-related apparatus such as the blood pressure monitor) is included as the type of health-related apparatus to be connected to the portable terminal 2, the estimation unit 1012 estimates a higher degree of interest. In one example, the estimation unit 1012 may estimate the degree of interest by putting the number or type of health-related apparatuses into a predetermined calculation equation. In another example, the estimation unit 1012 may refer to a table in which a predetermined number of health-related apparatuses and the degree of interest are associated with each other, and estimate the degree of interest based on the number of health-related apparatuses. Similarly, the estimation unit 1012 may refer to a table in which a predetermined type of health-related apparatus and the degree of interest are associated with each other, and estimate the degree of interest based on the type of health-related apparatus. These tables are stored in the storage device 104.

The estimation unit 1012 outputs the information indicating the degree of interest of the subject to the selection unit 1013.

The selection unit 1013 will be described. The selection unit 1013 selects the health information to be provided to the subject based on the degree of interest of the subject, as exemplified below. First, the selection unit 1013 receives the information indicating the degree of interest of the subject from the estimation unit 1012. Next, the selection unit 1013 selects the health information from the storage device 104 based on the degree of interest of the subject. The selection unit 1013 selects the health information corresponding to the degree of interest of the subject from the storage device 104. The selection unit 1013 may select the health information with an appropriate degree of detail according to the degree of interest of the subject. For example, the selection unit 1013 can refer to a table in which a predetermined degree of interest and the health information are associated with each other, and execute the process for selecting the health information. The table is stored in the storage device 104. The selection unit 1013 refers to the table and selects the health information at a level corresponding to the degree of interest of the subject from the storage device 104. The selection unit 1013 outputs the subject's health information to the output unit 1014.

The output unit 1014 will be described. The output unit 1014 outputs the subject's health information as exemplified below. First, the output unit 1014 receives the subject's health information from the selection unit 1013. Next, the output unit 1014 outputs the health information to be provided to the subject to the portable terminal 2 associated with the subject via the communication interface 105. Thereby, the portable terminal 2 can display the health information on a display unit 207. The subject can confirm the health information appropriate for the subject on the portable terminal 2.

### Portable Terminal

### Hardware Configuration

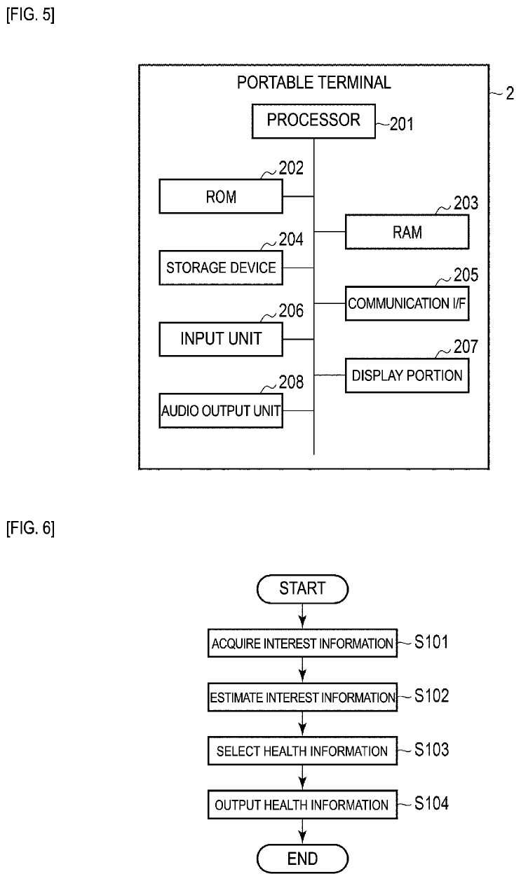

FIG. 5 is a block diagram illustrating a hardware configuration of the portable terminal 2.

The portable terminal 2 includes a processor 201, a ROM 202, a RAM 203, a storage device 204, a communication interface 205, an input unit 206, the display unit 207, and an audio output unit 208. The elements are electrically connected to each other. Note that, in FIG. 5, the communication interface is described as “communication I/F”.

The processor 201 controls each component of the portable terminal 2. For example, the processor 201 is a CPU, but is not limited thereto. The processor 201 deploys a program stored in the storage device 204 into the RAM 203 in order to execute the portable terminal 2. Then, the processor 201 interprets and executes the program deployed in the RAM 203, so that the processor 201 can execute various operations.

The storage device 204 is a so-called auxiliary storage device. For example, the storage device 204 is, but not limited to, a semiconductor memory such as an embedded or external flash memory. The storage device 204 stores a program that is executed by the processor 201. Note that the program may be stored in the storage device 204 in advance. The program may be downloaded to the portable terminal 2 via the network. The program may be stored and distributed on a non-transitory computer-readable medium.

The storage device 204 stores the interest information. The interest information is obtained by processing by the processor 201. For example, the processor 201 obtains the number of accesses or the frequency of accesses to the health-related web pages by referring to the history of access to the web pages. The processor 201 stores the information indicating the number of accesses or frequency of accesses to the health-related web pages, in the storage device 204. For example, the processor 201 obtains the number of health-related web pages or applications registered in the portable terminal 2 by referring to the URLs of the web pages registered in the browser. The processor 201 stores the information indicating the number of health-related web pages or applications registered in the portable terminal 2, in the storage device 204. For example, the processor 201 obtains the number of health-related apparatuses to be connected to the portable terminal 2 by referring to the number of applications installed in the portable terminal 2. The processor 201 obtains the type of health-related apparatus to be connected to the portable terminal 2 by referring to the type of application installed in the portable terminal 2. The processor 201 stores information indicating the number or type of health-related apparatuses to be connected to the portable terminal 2, in the storage device 204.

The communication interface 205 is an interface that communicably connects the portable terminal 2 to another device. The communication interface 205 includes a module for a short-range wireless communication system. The short-range wireless communication system is, for example, a communication system by Bluetooth (registered trademark), but is not limited thereto. The communication interface 205 can communicate directly with the blood pressure monitor 3 and the scale 4 by using the short-range wireless communication system. Further, the communication interface 205 includes modules for various types of wireless communication systems for mobile communication (3G, 4G, or the like), WLAN, and the like. In this case, the communication interface 205 can communicate with the server 1 by using various types of wireless communication systems.

The input unit 206 is a device that receives an instruction to be inputted by the subject. For example, the input unit 206 includes, but is not limited to, a touch panel and operation keys. The input unit 206 outputs a signal in accordance with the instruction inputted by the subject to the processor 201. Note that the input unit 206 is not limited to the device that receives the instruction by input based on the operation by the subject, and may be a device that receives an instruction by input based on the voice of the subject.

The display unit 207 is a device that displays information. For example, the display unit 207 is a liquid crystal display, but is not limited thereto.

The audio output unit 208 is a device that outputs sound. For example, the audio output unit 208 is a speaker, but is not limited thereto.

Note that, with regard to a specific hardware configuration of the portable terminal 2, components can be omitted, replaced, and added as appropriate according to the embodiment. For example, the portable terminal 2 may include a plurality of processors.

### 3 Operation Example

### Server

### Information Output Operation

FIG. 6 is an exemplary flowchart showing an information output operation in the server 1. Note that the processing procedure described below is merely an example, and each process may be changed to the extent possible. Further, for the processing procedure described below, steps can be omitted, replaced, and added as appropriate.

The acquisition unit 1011 acquires the interest information (step S101). In step S101, the acquisition unit 1011 acquires the interest information of the subject from the storage device 104 as exemplified. The acquisition unit 1011 outputs the interest information to the estimation unit 1012.

The estimation unit 1012 estimates the degree of interest (step S102). In step S102, the estimation unit 1012 estimates the subject's degree of interest in health based on the interest information, as exemplified. The estimation unit 1012 outputs the information indicating the degree of interest to the selection unit 1013.

The selection unit 1013 selects the health information (step S103). In step S103, the selection unit 1013 selects the health information to be provided to the subject based on the degree of interest of the subject, as exemplified. For example, the selection unit 1013 selects the health information with the appropriate degree of detail according to the degree of interest. The selection unit 1013 outputs the health information to the output unit 1014.

The output unit 1014 outputs the health information (step S104). In step S104, the output 1014 outputs the health information to be provided to the subject to the portable terminal 2 associated with the subject via the communication interface 105, as exemplified.

The processor 101 may repeatedly execute the above-described information output operation every time the interest information of each person is acquired from the storage device 104.

Note that in step S102, the estimation unit 1012 may estimate the degree of interest of the subject based on the health-related use by the portable terminal 2 associated with the subject, as described above. In the example, the interest information includes the usage information.

Note that, in step S102, the estimation unit 1012 may estimate the degree of interest in health based on the number of accesses or the frequency of accesses to the health-related web pages from the portable terminal 2, as described above. In the example, the usage information includes the information indicating the number of accesses or the frequency of accesses to the health-related web pages from the portable terminal 2.

Note that, in step S102, the estimation unit 1012 may estimate the degree of interest in health based on the number of health-related web pages or applications registered in the portable terminal 2, as described above. In the example, the usage information includes the information indicating the number of health-related web pages or applications registered in the portable terminal 2.

Note that, in step S102, the estimation unit 1012 may estimate the degree of interest in health based on the number or type of health-related apparatuses to be connected to the portable terminal 2, as described above. In the example, the usage information includes the information indicating the number or type of health-related apparatuses to be connected to the portable terminal 2.

Note that, in step S102, the estimation unit 1012 may create the history indicating the shift of the degree of interest according to the estimation of the degree of interest based on the health-related use by the portable terminal 2 associated with the subject. The history associates the estimated date and time of the degree of interest with the level of the degree of interest. The history may include identification information of the portable terminal 2 associated with the history. The history may include identification information of the subject associated with the history. The identification information of the portable terminal 2 and the identification information of the subject may be included in the interest information that the server 1 receives from the portable terminal 2. The estimation unit 1012 may store the history in the storage device 104. When the estimation unit 1012 creates the history, in step S103, the selection unit 1013 selects the health information as follows.

In one example, the selection unit 1013 detects the highest level of the degree of interest indicated in the history according to the estimation of the degree of interest based on the health-related use by the portable terminal 2 associated with the subject. For example, the selection unit 1013 detects the highest level of the degree of interest among a plurality of levels of the degree of interest included in the history. Next, the selection unit 1013 selects the health information at the level corresponding to the highest level of the degree of interest. For example, selection unit 1013 refers to the table, stored in storage 104, in which the predetermined degree of interest and the health information are associated with each other, and selects the health information at the level corresponding to the highest level of the degree of interest.

In another example, the 1013 determines whether the subject associated with the portable terminal 2 is the same as before according to the estimation of the degree of interest based on the health-related use by the portable terminal 2 associated with the subject. For example, the selection unit 1013 refers to the identification information of the portable terminal 2 and the identification information of the subject included in the interest information that is a basis for estimating the degree of interest. The selection unit 1013 acquires the history associated with the identification information of the portable terminal 2 included in the interest information from the storage device 104. The selection unit 1013 compares the identification information of the subject included in the interest information with the identification information of the subject included in the history. When the identification information of the subject included in the interest information corresponds to the identification information of the subject included in the history, the selection unit 1013 determines that the subject associated with the portable terminal 2 is the same as before. On the other hand, when the identification information of the subject included in the interest information does not correspond to the identification information of the subject included in the history, the selection unit 1013 determines that the subject associated with the portable terminal 2 is not the same as before. Accordingly, the selection unit 1013 can determine whether the subject is the same as before or the subject is not the same as before. Next, the selection unit 1013 detects the highest level of the degree of interest indicated in the history according to the determination result indicating that the subject is the same as before. For example, the selection unit 1013 detects the highest level of the degree of interest among a plurality of levels of the degree of interest included in the history. Next, the selection unit 1013 selects the health information at the level corresponding to the highest level of the degree of interest. On the other hand, the selection unit 1013 selects the health information at or below the level corresponding to the degree of interest estimated by the estimation unit 1012 based on the health-related use, according to the determination result indicating that the subject is not the same as before. For example, the selection unit 1013 refers the table, stored in the storage device 104, in which the predetermined degree of interest and the health information are associated with each other, and selects the health information at or below the level corresponding to the degree of interest estimated by the estimation unit 1012. Note that the selection unit 1013 may select either the health information at the level corresponding to the degree of interest or the health information below the level corresponding to the degree of interest. The criteria for selection can be arbitrarily set.

Note that, as described above, the server 1 acquires the interest information of the subject from the portable terminal 2, and outputs the health information to the portable terminal 2, but the information acquisition destination and output destination are not limited to the mobile terminal 2. The server 1 may acquire the interest information of the subject from a first external apparatus and output the health information to a second external apparatus different from the first external apparatus. Accordingly, the acquisition unit 1011 acquires the interest information from the first external apparatus. The output unit 1014 outputs the health information to the second external apparatus. The first external apparatus receives the health-related content spoken by the subject and generates the interest information based on the health-related content spoken by the subject. The first external apparatus transmits the interest information to the server 1. For example, the first external apparatus is, but not limited to, an apparatus such as an AI speaker or a smart speaker. For example, the second external apparatus is, but is not limited to, the portable terminal 2.

### 4 Action and Effect

As described above, in the present embodiment, the server 1 can estimate the degree of interest in health based on the interest information of the subject and output the health information based on the degree of interest.

The desired content of health information differs between those who have a high degree of interest in health and those who have a low degree of interest in health. The server 1 can provide the health information appropriate for the subject based on the degree of interest of the subject. The subject can effectively utilize the health information appropriate for the subject.

Further, in the present embodiment, the server 1 can estimate the degree of interest in health based on the health-related use by the portable terminal 2 associated with the subject.

People with the high degree of interest in health tend to use the portable terminals 2 for variety of purposes for their health. On the other hand, people with the low degree of interest in health tend to rarely use the mobile terminals 2 for their health. The server 1 can improve the estimation accuracy of the degree of interest in health by referring to the actual state of health-related uses by the portable terminal 2.

Further, in the present embodiment, the server 1 can estimate the degree of interest in health based on the number of accesses or the frequency of accesses to the health-related web pages from the portable terminal 2.

People with the high degree of interest in health tend to access health-related web pages for health many times using the portable terminals 2. On the other hand, people with the low degree of interest in health tend to rarely access health-related web pages using portable terminals 2. The server 1 can improve the estimation accuracy of the degree of interest in health by referring to the number of accesses or the frequency of accesses to the health-related web pages from the portable terminal 2.

Further, in the present embodiment, the server 1 can estimate the degree of interest in health based on the number of health-related web pages or applications registered in the portable terminal 2.

People with the high degree of interest in health tend to register many URLs of health-related web pages in the browser used on the portable terminals 2. Also, people with the high degree of interest in health tend to install many health-related applications on the portable terminals 2. On the other hand, people with the low degree of interest in health tend to rarely register the URLs of health-related web pages in the browser used in the portable terminals 2. Also, people with the low degree of interest in health tend to rarely install health-related applications on the portable terminals 2. The server 1 can improve the estimation accuracy of the degree of interest in health by referring to the number of health-related web pages or applications registered in the portable terminal 2.

Further, in the present embodiment, the server 1 can estimate the degree of interest in health based on the number or type of health-related apparatuses to be connected to the portable terminal 2.

People with the high degree of interest in health tend to connect many health-related apparatuses to the portable terminals 2. Also, people with the high degree of interest in health tend to connect not only health-related apparatuses, such as the scale 4, that are widely used in homes, but also medical health-related apparatuses, such as the blood pressure monitor 3, to the portable terminals 2. It can be said that the medical health-related apparatuses, such as the blood pressure monitor 3, are apparatuses for people with high degree of interest in health than the health-related apparatuses, such as the scale 4, that are widely used in homes. On the other hand, people with the low degree of interest in health tend to rarely connect health-related apparatuses to the portable terminals 2. Also, people with the low degree of interest in health tend to connect health-related apparatuses, such as the scale 4, that are widely used in homes to the portable terminals 2, but not connect medical health apparatuses, such as the blood pressure monitor 3, to the portable terminals 2. The server 1 can improve the estimation accuracy of the degree of interest in health by referring to the number or type of health-related apparatuses connected to the portable terminal 2.

In addition, in the present embodiment, the server 1 can detect the highest level of the degree of interest from the history and select the health information at the level corresponding to the highest level of the degree of interest.

Even when the subject's interest in health is lower than before, the subject's level of knowledge regarding health remains high. The server 1, by referring to the history indicating the shift of the degree of interest, can provide the health information with the degree of detail appropriate for the subject without degrading the quality of the health information as compared with the health information provided to the subject in the past. Since the subject receives the health information at the level that the subject can understand, the subject can effectively utilize the health information.

Further, in the present embodiment, the server 1 can detect the highest level of the degree of interest from the history according to the determination result indicating that the subject associated with the portable terminal 2 is the same as before, and select the health information at the level corresponding to the highest level of the degree of interest. The server 1 can select the health information at or below the level corresponding to the estimated degree of interest according to the determination result indicating that the subject associated with the portable terminal 2 is not the same as before.

The subject (owner) associated with the portable terminal 2 may change due to the transfer of the portable terminal 2 or the like. The knowledge level of the current subject associated with the portable terminal 2 may be lower than the knowledge level of another subject previously associated with the portable terminal 2. Thus, the current subject associated with the portable terminal 2 may not be able to understand the same level of health information that was previously provided to the other subject. The current subject associated with the portable terminal 2 preferably receives health information at or below the level corresponding to the current subject's degree of interest. The server 1 can provide the health information with the degree of detail appropriate for the subject by changing the level of health information that is provided to the subject according to whether the subject associated with the portable terminal 2 is the same as before.

Further, in the present embodiment, the server 1 can acquire the interest information from the first external apparatus and output the health information to the second external apparatus different from the first external apparatus.

For example, the subject may want to input the health-related content into the first external apparatus such as the AI speaker or the smart speaker by voice, and confirm the health information on the second external apparatus such as the subject's portable terminal 2. With the separate apparatuses, the subject can input the health-related content and confirm the health information, thereby improving the convenience of the subject.

In addition, in the present embodiment, the server 1 can select the health information with the appropriate degree of detail according to the degree of interest in health.

The higher the subject's interest in health, the more likely the subject wants more detailed health information. The server 1 can provide the health information with the degree of detail appropriate for the subject based on the degree of interest of the subject. The subject can effectively utilize the health information appropriate for the subject.

### 5 Modified Examples

### 5-1 Modified Example 1

In the present embodiment, the example in which the server 1 acquires the interest information and estimates the degree of interest based on the interest information has been described, but the present invention is not limited to this example. The processor 201 of the portable terminal 2 may estimate the degree of interest based on the interest information. In the example, the processor 201 of the portable terminal 2 implements an acquisition unit corresponding to the acquisition unit 1011 and an estimation unit corresponding to the estimation unit 1012 to be implemented on the server 1. The portable terminal 2 transmits a request for health information to the server 1. The request for health information includes information indicating the degree of interest. The server 1 receives the request for health information from the portable terminal 2. The server 1 refers to the information indicating the degree of interest included in the request for health information. The server 1 transmits the health information based on the degree of interest to the portable terminal 2 as a response to the request for health information.

### 5-2 Modified Example 2

In short, the invention is not limited to the present embodiment described above and can be embodied by modifying the components in an implementation stage in a range without departing from the gist thereof. Further, various inventions can be formed by appropriately combining a plurality of components disclosed in the present embodiment. For example, some components may be omitted from the entire components shown in the present embodiment. Furthermore, the components common to different embodiments may be combined appropriately.

### 6 Supplementary Note

In addition to the claims, some or all of the present embodiment may be described as shown in the following supplementary note, but the invention is not limited thereto.

### Supplementary Note

A server (1) includes an acquisition unit (1011) configured to acquire first information indicating a subject's interest in health, an estimation unit (1012) configured to estimate the subject's degree of interest in health based on the first information, a selection unit (1013) configured to select second information regarding health to be provided to the subject based on the degree of interest, and an output unit (1014) configured to output the second information.

### REFERENCE SIGNS LIST

1 Server
2 Portable terminal
3 Blood pressure monitor
4 Scale
100 Information distribution system
101 Processor
102 ROM
103 RAM
104 Storage device
105 Communication interface
201 Processor
202 ROM
203 RAM
204 Storage device
205 Communication interface
206 Input unit
207 Display unit
208 Audio output unit
1011 Acquisition unit
1012 Estimation unit
1013 Selection unit
1014 Output unit
A1 Server
A2 Portable terminal

1. An information processing device comprising:
one or more processors configured to:
acquire first information indicating a subject's interest in health, including information indicating the number of health-related apparatuses to be connected to a terminal associated with the subject;
estimate a subject's degree of interest in health based on the number of health-related apparatuses;
select second information regarding health to be provided to the subject based on the degree of interest; and
output the second information.

2. The information processing device according to claim 1, wherein
the first information includes information indicating the number of accesses or a frequency of accesses to a health-related web page from the terminal, and
the one or more processors is further configured to estimate the degree of interest based on the number of accesses or the frequency of accesses.

3. The information processing device according to claim 1, wherein
the first information includes information indicating the number of web pages or applications regarding health registered in the terminal, and
the one or more processors is further configured to estimate the degree of interest based on the number of web pages or applications.

4. The information processing device according to claim 2, wherein
the one or more processors is further configured to:
create a history indicating a shift of the degree of interest according to the estimation of the degree of interest based on the number of health-related apparatuses, and
detect a highest level of the degree of interest indicated in the history according to the estimation of the degree of interest based on the number of health-related apparatuses, and select the second information at a level corresponding to the highest level of the degree of interest.

5. The information processing device according to claim 2, wherein
the one or more processors is further configured to:
create a history indicating a shift of the degree of interest according to the estimation of the degree of interest based on the number of health-related apparatuses, and
determine whether the subject associated with the terminal is the same as before according to the estimation of the degree of interest based on the number of health-related apparatuses, and when a determination result indicates that the subject is the same as before, detect a highest level of the degree of interest indicated in the history and select the second information at a level corresponding to the highest level of the degree of interest, and when a determination result indicates that the subject is not the same as before, select the second information at or below the level corresponding to the degree of interest estimated based on the number of health-related apparatuses.

6. The information processing device according to claim 1, wherein
the one or more processors is further configured to:
acquire the first information from a first external apparatus, and
output the second information to a second external apparatus different from the first external apparatus.

7. The information processing device according to claim 1, wherein
the one or more processors is further configured to select the second information with an appropriate degree of detail according to the degree of interest.

8. An information processing method comprising:
acquiring first information indicating a subject's interest in health, including information indicating the number of health-related apparatuses to be connected to a terminal associated with the subject;
estimating a subject's degree of interest in health based on the number of health-related apparatuses;
selecting second information regarding health to be provided to the subject based on the degree of interest; and
outputting the second information.

9. A non-transitory recording medium on which a program for information processing is recorded, the program causes a computer to execute functions included in the information processing device according to claim 1.
